(19) United States
(12) Patent Application Publication
(10) Pub. No.: US 2016/0195257 A1
(43) Pub. Date: Jul. 7, 2016

(54) WALL-MOUNT AROMA DIFFUSING NIGHT LAMP ASSEMBLY

(71) Applicant: Ming Jen Hsiao, Miaoli County (TW)

(72) Inventor: Ming Jen Hsiao, Miaoli County (TW)

(21) Appl. No.: 14/879,675

(22) Filed: Oct. 9, 2015

Related U.S. Application Data
(63) Continuation-in-part of application No. 14/589,129, filed on Jan. 5, 2015.

(51) Int. Cl. F21V 33/00; A61L 9/03; F21V 21/30; F21V 23/02; F21V 8/00; F21V 23/06

(52) U.S. Cl. CPC F21V 33/0004; G02B 6/0073; F21V 23/06; F21V 21/30; F21V 23/02; A61L 9/03; F21Y 2101/02; F21W 2131/30

(57) ABSTRACT

A wall-mount aroma-diffusing night lamp assembly includes an electrical plug angularly adjustably connected to a housing, a heating element electrically connected to the electrical plug, a heat transfer member kept in contact with the heating element, a light-emitting device, a retaining device fastened to the housing, and a light guide plate detachably connected to the retaining device around a part of the periphery of the housing a uniform glowing effect during operation of the light-emitting device, and a disposable aroma capsule placed on the heat transfer member and heatable to release a pleasant smell.

### CROSS-REFERENCE TO RELATED APPLICATION

The present application is a continuation-in-part of U.S. application Ser. No. 14/589,129 filed on Jan. 5, 2015, now pending.

### BACKGROUND OF THE INVENTION

1. Field of the Invention

This invention relates night lamp technology and more particularly, to a wall-mount aroma diffusing night lamp assembly, which ensures a high level of wall-mount.

1. Description of the Related Art

Various aroma diffusing designs are known. Exemplars are seen in U.S. Pat. No. 8,066,420; U.S. Pat. No. 8,262,277; US2012/0024837A1. These designs commonly include a power source and a heat source. In application, the user needs to prepare a particular container for holding an aromatic wax or essential oil, and then to carefully pick up the aromatic wax or essential oil from the container and to put the aromatic wax or essential oil in an accommodation chamber for heating by the heat source, for example, the radiating heat energy of the lamp bulb. Further, when the aromatic wax or essential oil is used up, the user needs to clean the accommodation chamber, avoiding mixing of different aromatic substances or smells. Further, if the accommodation chamber is made of a fragile material, such as ceramic or glass, the accommodation chamber can be accidentally broken during cleaning. Further, some light-emitting aroma diffusers emit light through holes in the periphery of a housing or the light transmission wall of a lampshade. However, the visual effect thus created is still not very attractive. Further, conventional wall-mount aroma-diffusing lamp designs are configured to emit light in all directions through the periphery thereof. However, when mounted in an electrical outlet in the wall, the back side of the lamp faces toward the wall, and thus, a part of the emitted light is blocked by the wall. Thus, the conventional wall-mount aroma-diffusing lamp designs waste much electrical energy for emitting light in unnecessary directions. Further, conventional wall-mount aroma-diffusing lamp designs cannot concentrate light to enhance luminous brightness.

### SUMMARY OF THE INVENTION

The present invention has been accomplished under the circumstances in view. It is therefore the main object of the present invention to provide a wall-mount aroma-diffusing night lamp assembly, which concentrates light radiation and saves power consumption.

It is another object of the present invention to provide a wall-mount aroma-diffusing night lamp assembly, which facilitates mounting and dismounting.

It is another object of the present invention to provide a wall-mount aroma-diffusing night lamp assembly, which uses a disposable aroma capsule for releasing a pleasant smell.

To achieve these and other objects of the present invention, a wall-mount aroma-diffusing night lamp assembly comprises an electrical plug for connection to a power source, a housing joined to the electrical plug and defining an opening in a top side thereof, a heating element mounted inside the housing and electrically connected to the electrical plug, a heat transfer member mounted inside the housing and kept in contact with the heating element, a light-emitting device mounted at one side inside the housing and electrically connected to the electrical plug, a retaining device joined to the housing and comprising an engagement groove, and a light guide plate comprising an engagement flange detachably engageable in the engagement groove of the retaining device. The light-emitting device can be a low luminous power and low power consumption LED lamp for emitting light in direction reversed to the wall of the electrical wall outlet (receptacle) to which the electrical plug is connected, concentrating light radiation and saving power consumption.

Further, the light guide plate of the wall-mount aroma-diffusing night lamp assembly is not hindered by the wall of the electrical wall outlet, and adapted for guiding the emitted light from the light-emitting device toward the front side of the wall-mount aroma-diffusing night lamp assembly away from the wall of the electrical wall outlet (receptacle). Thus, the light-emitting device can be a low luminous power and low power consumption LED lamp for emitting light in direction reversed to the wall of the electrical wall outlet (receptacle), concentrating light radiation and saving power consumption.

The engagement flange of the retaining device is detachably engageable in the engagement groove of the retaining device so that the light guide plate can be conveniently and detachably fastened to the retaining device for creating a uniform glowing effect around the wall-mount aroma-diffusing night lamp assembly.

Preferably, the wall-mount aroma-diffusing night lamp assembly further comprises an aroma capsule inserted through the opening of the housing and placed on the heat transfer member. Thus, the heat emitted by the heating element can be transferred through the heat transfer member to the aroma capsule, causing the aroma capsule to release a pleasant smell. The heat transfer member can be a metal or ceramic-based heat spreader. The aroma capsule has an aroma wax or essential oil contained therein heatable to release a pleasant smell. After the use, the aroma capsule can be thrown away or recycled. Replacing the aroma capsule does not need to clean the housing of the wall-mount aroma-diffusing night lamp assembly.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is a sectional elevational view of a wall-mount aroma-diffusing night lamp assembly in accordance with the present invention.

FIG. 2 is an exploded perspective view of the wall-mount aroma-diffusing night lamp assembly in accordance with the present invention.

FIG. 3 is another exploded view of the wall-mount aroma-diffusing night lamp assembly in accordance with the present invention.

FIG. 4 is sectional side view of the wall-mount aroma-diffusing night lamp assembly in accordance with the present invention.

### DETAILED DESCRIPTION OF THE PREFERRED EMBODIMENT

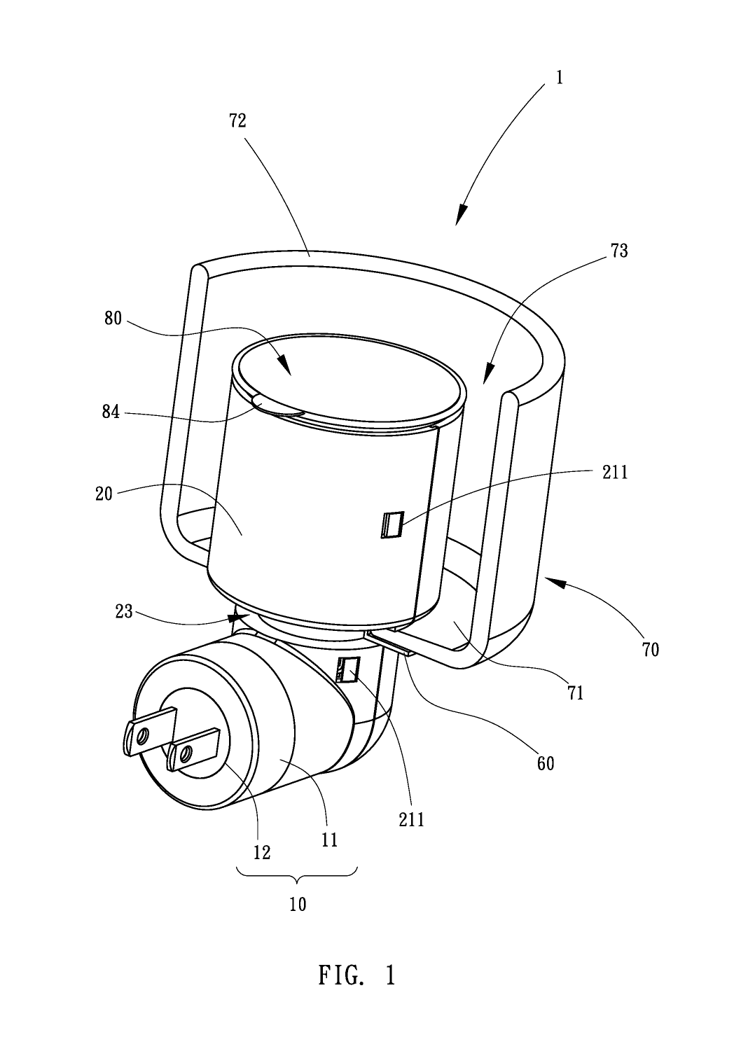

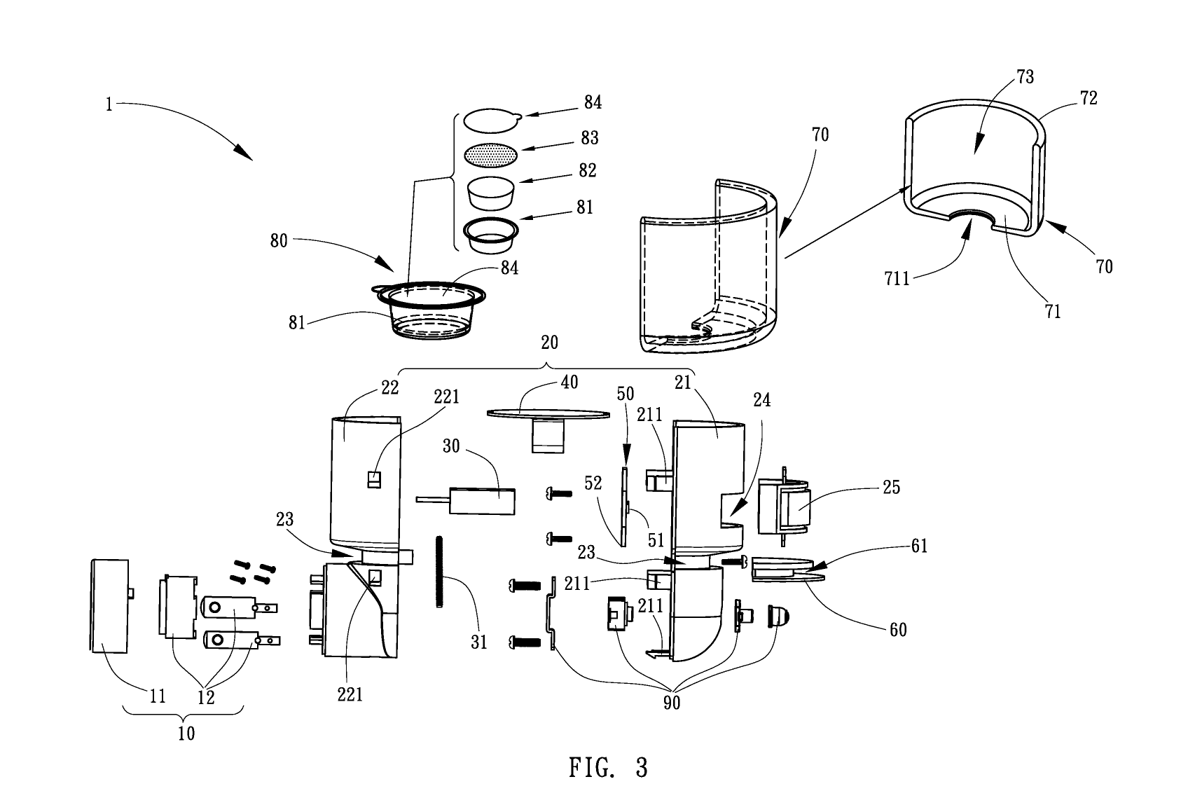

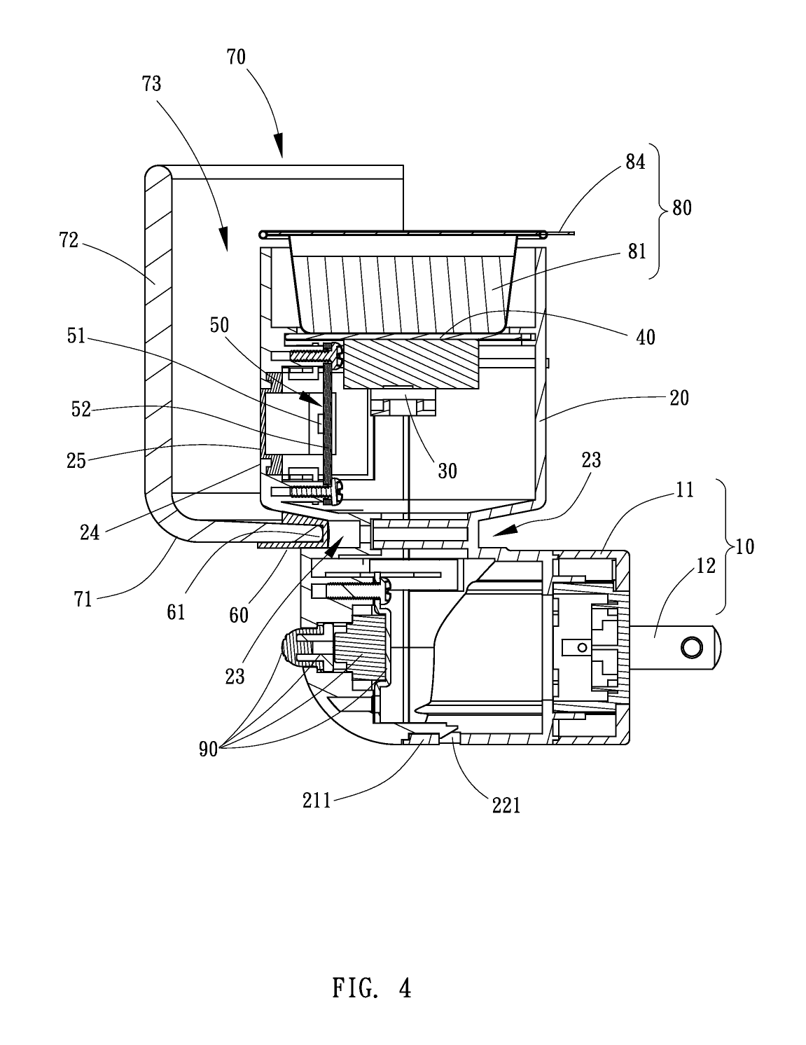

Referring to FIGS. 1-4, a wall-mount aroma-diffusing night lamp assembly in accordance with the present invention is shown.

The wall-mount aroma-diffusing night lamp assembly comprises an electrical plug 10 for connection to power source, a housing 20 fastened to the electrical plug 10 and defining an opening 26 in a top side thereof, a heating element 30 mounted in the housing 20 and electrically connected to the electrical plug 10, a heat transfer member 40 mounted in the housing 20 with one side thereof kept in contact with the heating element 30, a light-emitting device 50 mounted in the housing 20 at one side and electrically connected to the electrical plug 10 to obtain the necessary working voltage from the electrical plug 10, a retaining device 60 fastened to the housing 20 and providing an engagement groove 61, a light guide plate 70 having an engagement flange 71 forced into engagement with the engagement groove 61 of the retaining device 60.

The electrical plug 10 is mountable in an electrical wall outlet (receptacle) for power input. Further, the light-emitting device 50 is configured to emit light in direction toward a front side of the wall-mount aroma-diffusing night lamp assembly 1, i.e., the side of the wall-mount aroma-diffusing night lamp assembly 1 that is not hindered by the wall of the electrical wall outlet (receptacle). Further, the light guide plate 70 of the wall-mount aroma-diffusing night lamp assembly 1 is also not hindered by the wall of the electrical wall outlet (receptacle), and adapted for guiding the emitted light from the light-emitting device 50 toward the front side of the wall-mount aroma-diffusing night lamp assembly 1 away from the wall of the electrical wall outlet (receptacle). Thus, the light-emitting device 50 can be a low luminous power and low power consumption LED lamp for emitting light in direction reversed to the wall of the electrical wall outlet (receptacle), concentrating light radiation and saving power consumption.

The mating connection structure between the light guide plate 70 and the retaining device 60 is detachable, facilitating mounting, dismounting and displacement of the light guide plate 70.

Further, an aroma capsule 80 can be placed in the housing 20 in contact with the heat transfer member 40. When the heating element 30 is driven to emit heat, the emitted thermal energy is transferred by the heat transfer member 40 to the aroma capsule 80, causing the aroma capsule 80 to diffuse an aromatic smell. The aroma capsule 80 can be thrown away or recycled after use. It is convenient to replace the aroma capsule 80. When replacing the aroma capsule 80, the user needs not to clean the wall-mount aroma-diffusing night lamp assembly. The light guide plate 70 receives and guides the emitted light from the light-emitting device 50, creating a uniform glowing effect around the wall-mount aroma-diffusing night lamp assembly 1 to match with the overall atmosphere of the fragrance exerted by the aroma capsule 80.

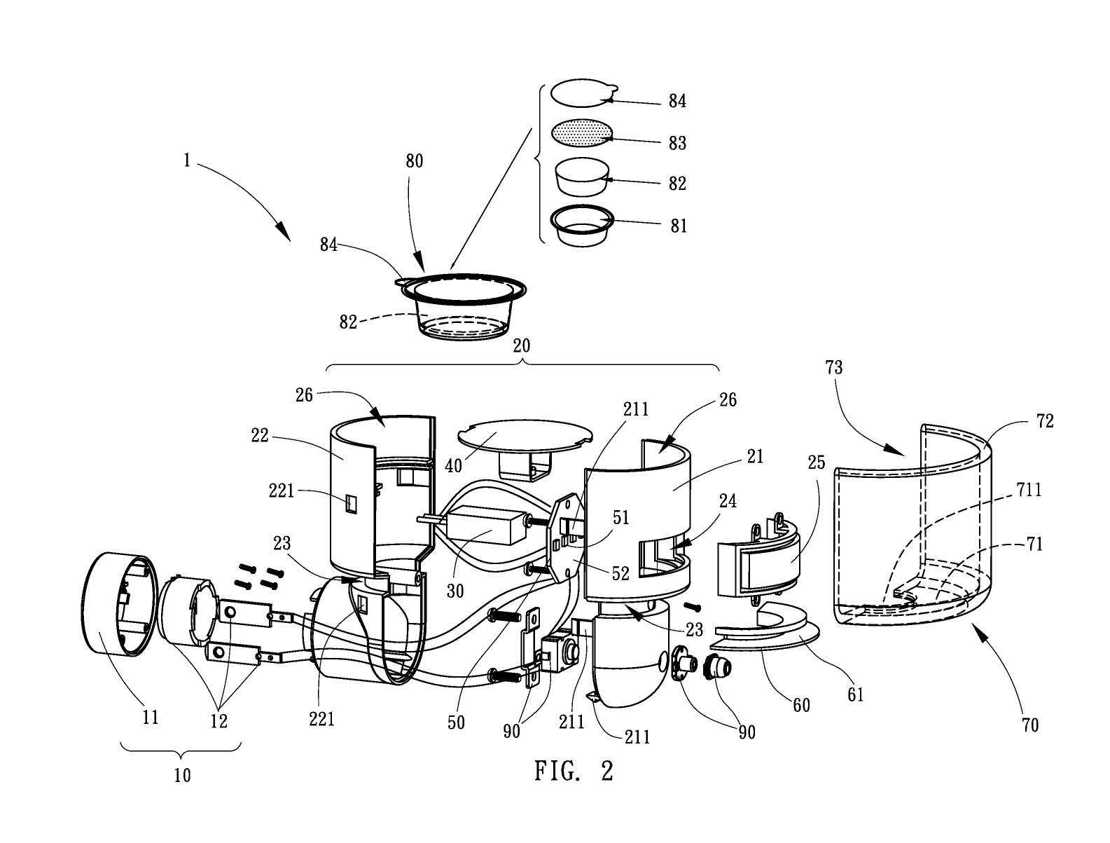

Referring to FIGS. 2-4 again, the aroma capsule 80 used in the wall-mount aroma-diffusing night lamp assembly 1 must be arranged to face upward. In order to fit different installation requirements for application in different countries, the electrical plug 10 is selectively positioned in one of a series of angular positions relative to the housing 20. The electrical plug 10 comprises a holder base 11 and a rotary plug body 12. The holder base 11 is fastened to the housing 20. The rotary plug body 12 is mounted in the holder base 11 and selectively and rotatably positionable in one of a series of equiangularly spaced positions. Thus, the rotary plug body 12 can be rotated relative to the holder base 11 and the housing 20 and positioned in such a position that the aroma capsule 80 is kept to face upward. In this embodiment, the rotary plug body 12 can be rotated either clockwise or counter-clockwise relative to the holder base 11 and the housing 20 and positioned in, for example, 90°, 180°, 270° and 360° to fit different installation requirements.

Further, the heating element 30 can be cement resistor, thermistor or PTC (positive temperature coefficient) heating member.

Preferably, the housing 20 comprises a first housing member 21 and a second housing member 22. The first housing member 21 comprises a plurality of hooks 211. The second housing member 22 comprises a plurality of hook holes 221 respectively forced into engagement with the hooks 211 of the first housing member 21. Thus, the first housing member 21 and the second housing member 22 are fastened together, forming the housing 20.

Preferably, the housing 20 comprises a mounting groove 23 extending around the periphery thereof. The retaining device 60 is engaged into the mounting groove 23 and secured to the housing 20. The retaining device 60 simply needs to be arranged in the area of the wall-mount aroma-diffusing night lamp assembly 1 that is kept away from the wall of the electrical wall outlet (receptacle), facilitating mounting and dismounting. In this embodiment, the retaining device 60 is arc-shaped, exhibiting a substantially C-shaped profile mating with the configuration of the housing 20.

Preferably, the housing 20 comprises a side hole 24 located at one lateral side thereof corresponding to the light-emitting device 50, and a light transmission window 25 mounted in the side hole 24. Thus, the light emitted by the light-emitting device 50 can go through the light transmission window 25 toward the light guide plate 70, avoiding waste of light source and saving power consumption.

Preferably, a support member 31 is mounted in the housing 20 to support the heating element 30 inside the housing 20.

Preferably, the light-emitting device 50 comprises a plurality of light-emitting components 51 and a circuit board 52. The light-emitting components 51 are electrically connected to the circuit board 52. In this embodiment, the light-emitting components 51 are light-emitting diodes (LEDs). The circuit board 52 is fastened to the inside of the housing 20 by screws, and electrically connected to the electrical plug 10 to provide the necessary working voltage to the light-emitting components 51. Further, the circuit board 52 is configured to control the light-emitting components (LEDs) 51 to emit different colors of light or to flash, causing the light guide plate 70 to create a uniform glowing effect around the wall-mount aroma-diffusing night lamp assembly 1. Thus, the wall-mount aroma-diffusing night lamp assembly 1 of the invention is capable of providing a decorative effect totally unlike conventional aroma diffusers.

Further, the circuit board 52 comprises a rectifier circuit for rectifying input AC power supply into the desired DC power supply for working. Thus, the electrical plug 10 can be directly connected to a city power supply outlet without an external power transformer, adapter or rectifier, bringing convenience. Further, the circuit board 52 is mounted inside the housing 20, assuring a high level of safety.

As described above, the light guide plate 70 of the wall-mount aroma-diffusing night lamp assembly 1 is mounted around a part of the periphery of the housing 20 that is not hindered by the wall of the electrical wall outlet (receptacle). Preferably, the engagement flange 71 of the light guide plate 70 has a notch 711 mating with the configuration of the engagement groove 61 of the retaining device 60 so that the engagement flange 71 can be conveniently and positively engaged into the engagement groove 61 of the retaining device 60. The light guide plate 70 further comprises an arc-shaped enclosed body portion 72 upwardly extended from one side of the engagement flange 71, and an open side 73 opposing the enclosed body portion 72.

Preferably, the aroma capsule 80 comprises a disposable heat-transfer container 81 placed on the heat transfer member 40, and an aromatic substance 82 such as aromatic wax or essential oil contained in the disposable heat-transfer container 81. The disposable heat-transfer container 81 is kept in contact with the heat transfer member 40 to transfer heat to the aromatic substance 82, causing the aromatic substance 82 to release a pleasant smell. After use, the disposable heat-transfer container 81 can be thrown away or recycled.

Preferably, the aroma capsule 80 further comprises a aroma capsule 8, which comprises a breathing film 83 covered over a top open side of the disposable heat-transfer container 81, and a sealing film 84 bonded to the disposable heat-transfer container 81 over the breathing film 83 to seal the solid aromatic substance 82 in the disposable heat-transfer container 81 and to maintain the quality of the solid aromatic substance 82.

Further, the breathing film 83 is selected from the material group of nonwoven fabric, fiber cloth, woven fabric, porous plastic membrane and porous silicon rubber. When in use, the user simply needs to remove the sealing film 84 from the disposable heat-transfer container 81 without contacting the solid aromatic substance 82.

Preferably, the wall-mount aroma-diffusing night lamp assembly 1 further comprises a switch 90 electrically coupled with the electrical plug 10, the heating element 30 and the light-emitting device 50 and switchable to turn on/off the heating element 30 and the light-emitting device 50.

Although a particular embodiment of the invention has been described in detail for purposes of illustration, various modifications and enhancements may be made without departing from the spirit and scope of the invention. Accordingly, the invention is not to be limited except as by the appended claims. the invention is not to be limited except as by the appended claims.

What the invention claimed is:

1. A wall-mount aroma-diffusing night lamp assembly, comprising:
an electrical plug for connection to a power source;
a housing joined to said electrical plug, said housing comprising an opening located in a top side thereof;
a heating element mounted inside said housing and electrically connected to said electrical plug;
a heat transfer member mounted inside said housing and kept in contact with said heating element;
a light-emitting device mounted at one side inside said housing and electrically connected to said electrical plug;
a retaining device joined to said housing, said retaining device comprising an engagement groove;
a light guide plate comprising an engagement flange detachably engageable in said engagement groove of said retaining device.

2. The wall-mount aroma-diffusing night lamp assembly as claimed in claim 1, wherein said electrical plug comprises a holder base affixed to said housing, and a rotary plug body rotatably mounted in said holder base and positionable in one of a series of angular positions relative to said holder base.

3. The wall-mount aroma-diffusing night lamp assembly as claimed in claim 1, wherein said housing comprises a first housing member and a second housing member, said first housing member comprising at least one hook, said second housing member comprising at least one hook hole detachably connectable to said at least one hook of said first housing member.

4. The wall-mount aroma-diffusing night lamp assembly as claimed in claim 1, wherein said housing comprises a mounting groove extending around the periphery thereof; said retaining device is detachably fastenable to said mounting groove of said housing.

5. The wall-mount aroma-diffusing night lamp assembly as claimed in claim 1, wherein said retaining device is arc-shaped.

6. The wall-mount aroma-diffusing night lamp assembly as claimed in claim 4, wherein said retaining device is arc-shaped.

7. The wall-mount aroma-diffusing night lamp assembly as claimed in claim 1, wherein said housing further comprises a side hole facing toward said light-emitting device, and a light transmission window mounted in said side hole.

8. The wall-mount aroma-diffusing night lamp assembly as claimed in claim 1, wherein said light-emitting device comprises a circuit board and at least one light-emitting component electrically connected to said circuit board, said circuit board comprising a rectifier circuit adapted for rectifying AC power supply into DC power supply.

9. The wall-mount aroma-diffusing night lamp assembly as claimed in claim 1, wherein said engagement flange of said light guide plate comprises a notch mating with the configuration of said engagement groove of said retaining device.

10. The wall-mount aroma-diffusing night lamp assembly as claimed in claim 9, wherein said light guide plate further comprises an arc-shaped enclosed body portion upwardly extended from one side of said engagement flange, and an open side opposing said enclosed body portion.

11. The wall-mount aroma-diffusing night lamp assembly as claimed in any of claim 1, further comprising an aroma capsule inserted through said opening of said housing and placed on said heat transfer member, said aroma capsule comprising a disposable heat-transfer container, and aromatic substance contained in said disposable heat-transfer container.

12. The wall-mount aroma-diffusing night lamp assembly as claimed in any of claim 2, further comprising an aroma capsule inserted through said opening of said housing and placed on said heat transfer member, said aroma capsule comprising a disposable heat-transfer container, and aromatic substance contained in said disposable heat-transfer container.

13. The wall-mount aroma-diffusing night lamp assembly as claimed in any of claim 7, further comprising an aroma capsule inserted through said opening of said housing and placed on said heat transfer member, said aroma capsule comprising a disposable heat-transfer container, and aromatic substance contained in said disposable heat-transfer container.

14. The wall-mount aroma-diffusing night lamp assembly as claimed in any of claim 8, further comprising an aroma capsule inserted through said opening of said housing and placed on said heat transfer member, said aroma capsule comprising a disposable heat-transfer container, and aromatic substance contained in said disposable heat-transfer container.
